(19) United States
(12) Patent Application Publication
(10) Pub. No.: US 2025/0288341 A1
(43) Pub. Date: Sep. 18, 2025

(54) BONE CEMENT MIXING SYSTEM WITH PUNCTURE CONNECTION

(71) Applicant: Heraeus Medical GmbH, Wehrheim (DE)

(72) Inventors: Sebastian VOGT, Wehrheim (DE); Thomas KLUGE, Wehrheim (DE)

(21) Appl. No.: 19/075,511

(22) Filed: Mar. 10, 2025

(30) Foreign Application Priority Data
Mar. 15, 2024 (EP) 24163703.2

(51) Int. Cl. A61B 17/88

(52) U.S. Cl. CPC A61B 17/8833; A61B 17/8822; A61B 2017/8838

(57) ABSTRACT

A device for producing bone cement, comprising a base part which has a container for receiving a monomer liquid, a bottom part with a collection region, and a hollow mandrel with a tip, and a cartridge for receiving a PMMA bone cement powder, wherein the cartridge has a septum which delimits the cartridge toward the outside, wherein the device is designed and configured to form a fluid-conducting connection between the base part and the cartridge by the hollow mandrel piercing the septum so as to transfer a monomer liquid from the collection region into the cartridge.

### CROSS-REFERENCE TO RELATED APPLICATIONS

This application claims priority pursuant to 35 U.S.C. 119(a) to European Patent Application No. 24163703.2, filed Mar. 15, 2024, which application is incorporated herein by reference in its entirety.

### FIELD OF THE INVENTION

The present invention relates to the field of medical engineering, in particular to devices for producing PMMA bone cement and for storing components of said bone cement.

### BACKGROUND OF THE INVENTION

Polymethyl methacrylate (PMMA) bone cements stem from the fundamental work of CHARNLEY. Polymethyl methacrylate bone cements are usually composed of a liquid monomer component and a powder component. The monomer component may comprise, for example, the monomer methyl methacrylate and an activator (e.g., N,N-dimethyl-p-toluidine) dissolved therein. The powder component, also referred to as bone cement powder, can comprise one or more polymers producible on the basis of methyl methacrylate and comonomers, such as styrene, methyl acrylate or similar monomers, by polymerization, preferably suspension polymerization. The powder component may further comprise a radiopaque agent and an initiator such as dibenzoyl peroxide. When the powder component is mixed with the monomer component, a plastically deformable dough, the actual bone cement dough, may form as a result of the polymers of the powder component swelling in the methyl methacrylate. When the powder component is mixed with the monomer component, the activator N,N-dimethyl-p-toluidine, for example, reacts with dibenzoyl peroxide to form radicals. The radicals formed can initiate the radical polymerization of the methyl methacrylate. As the polymerization of the methyl methacrylate progresses, the viscosity of the cement dough increases until the cement dough solidifies.

Polymethyl methacrylate bone cements can be mixed in suitable mixing cups with the aid of spatulas by mixing the powder component with the monomer liquid. Air bubbles may be incorporated in the bone cement dough, which can negatively influence the mechanical properties of the hardened bone cement.

A plurality of vacuum cementing systems have been described for avoiding air inclusions in the bone cement dough, of which the following are mentioned by way of example: U.S. Pat. Nos. 6,033,105 A, 5,624,184 A, 4,671,263 A, 4,973,168 A, 5,100,241 A, WO 99/67015 A1, EP 1 020 167 A2, U.S. Pat. No. 5,586,821 A, EP 1 016 452 A2, DE 36 40 279 A1, WO 94/26403 A1, EP 1 005 901 A2, and U.S. Pat. No. 5,344,232 A.

Cementing systems in which both the cement powder and the monomer liquid are already packed in separate compartments of the mixing systems and are not mixed together in the cementing system until immediately before the cement is applied represent a development in cementing technology. Such closed “fully prepacked mixing systems” are described in patent documents EP0380867B1, EP0796653B1, EP0692229B1, U.S. Pat. Nos. 5,997,544A, 6,709,149B1, DE69812726T2 and U.S. Pat. No. 5,588,745A, WO9416951A1, DE19718648A1, EP1741413B1, EP3054880B1, DE102009031178B3, U.S. Pat. No. 8,662,736B2, EP2269718B1, EP2281532B1, and EP3093067B1.

Fully prepacked mixing systems encounter a few challenges. It is desirable to ensure safe, reproducible transfer of the monomer liquid to the polymethyl methacrylate bone cement powder and to avoid uncontrolled mixing of the components while they are being stored. It is advantageous if, during transport and storage of the mixing system, the cement powder cannot penetrate into the line means for transferring the monomer liquid. Otherwise, the line means may become blocked during transfer of the monomer as a result of the cement powder swelling and clumping. Blockage of the line means by swollen bone cement clumping could interrupt the transfer of the monomer liquid and prevent the cement components from mixing in the predetermined mixing ratio.

In the fully prepacked mixing systems according to EP0796653B1, U.S. Pat. No. 6,709,149B1, EP1741413B1, EP3054880B1, EP2269718B1 and EP2281532B1, the monomer is transferred by applying an external vacuum to the cartridge via a vacuum connection and a porous plate. This means that the monomer liquid is drawn into the cartridge by the vacuum applied. In the mixing system described in EP3093067B1, the monomer liquid can either be transferred to the cartridge by the action of a vacuum, or alternatively the monomer liquid can be transferred by being pushed by means of a manually operable pump piston integrated into the mixing system.

According to EP2281532B1, blockage of the line means for transferring the monomer liquid is avoided by using a mesh or porous disk permeable to the monomer liquid for blocking the cement powder particles, wherein the mesh or porous disk is part of a special nozzle that generates a sharp monomer liquid jet during the transfer, which displaces swollen cement particles so that the monomer liquid can be fully transferred into the bone cement powder.

EP1741413B1 discloses a method for bringing a powder and a liquid component into contact with one another, wherein a liquid container is opened by penetration of a cannula into a wall portion of the liquid container which closes a lateral hole in an inner tube element in the liquid container.

Patent document EP3636338B1 discloses a mixing system comprising a connecting cylinder configured to be selectively coupled to and removable from a mixing chamber, and wherein a valve is configured to seal the connection between the connecting cylinder and the valve when they are selectively coupled to one another, and is configured to seal the mixing chamber when the connecting cylinder is removed from the mixing chamber. EP3490700B1 discloses a similar mixing system.

### SUMMARY OF THE INVENTION

An object of the present invention is to solve one or more of the problems described above and further problems of the prior art. For example, the invention provides a device for storing and mixing bone cement, which allows simple and reliable mixing of a monomer liquid with a powder component of a PMMA bone cement so as to produce a ready-to-use PMMA bone cement dough. The devices according to the invention can comprise a plurality of parts which can be connected to one another and disconnected from one another again in a simple manner, thereby allowing a reliable and safe transfer of a monomer liquid from one part of the device to the other part of the device. A self-sealing connection system prevents monomer liquid from leaking out or contamination from entering. The device according to the invention can provide simple handling.

A monomer liquid and a powder component of PMMA bone cement can be separately stored, combined in a controlled manner and mixed together in the device. The device can perform these functions while securely placed on a support. The monomer liquid can be fully transferred, thereby preventing the device from being clogged up by clumps of powder.

These objects are achieved by the methods, devices, kits and medical uses described herein, in particular those which are described in the claims.

Preferred embodiments of the invention will be described below.

A first embodiment relates to a device for producing bone cement, comprising a base part which comprises a container for receiving a monomer liquid, a bottom part with a collection region, and a hollow mandrel with a tip, and a cartridge for receiving a PMMA bone cement powder, wherein the cartridge comprises a septum which delimits the cartridge toward the outside, wherein the device is designed and configured to form a fluid-conducting connection between the base part and the cartridge by the hollow mandrel piercing the septum so as to transfer a monomer liquid from the collection region into the cartridge.

A second embodiment relates to a device according to the first embodiment, wherein the base part further comprises a piston, wherein the piston comprises a passage for receiving the hollow mandrel, wherein, in a basic configuration of the device, the piston is configured to receive the tip of the hollow mandrel in the passage, and to release the tip of the hollow mandrel from the passage when the cartridge is pushed onto the piston.

A third embodiment relates to a device according to the second embodiment, wherein the base part comprises a spring which is arranged and configured for movably mounting the piston within the base part.

A fourth embodiment relates to a device according to either the second or the third embodiment, wherein the base part comprises a stop to limit the movement of the piston and/or the cartridge in the base part.

A fifth embodiment relates to a device according to any one of the preceding embodiments, wherein the device comprises a filter, wherein the filter is designed and configured to prevent particles from entering the cartridge from the base part.

A sixth embodiment relates to a device according to any one of the preceding embodiments, wherein the cartridge comprises a connection for connection to a vacuum source to enable a monomer liquid to be transferred from the collection region into the cartridge by means of a pressure difference.

A seventh embodiment relates to a device according to any one of the preceding embodiments, wherein the container comprises a carrier for receiving a glass ampule or a film pouch containing monomer liquid.

An eighth embodiment relates to a device according to the seventh embodiment, wherein the container comprises an ampule breaker, wherein the ampule breaker is configured to open a glass ampule within the container.

A ninth embodiment relates to a device according to any one of the preceding embodiments, wherein the ampule breaker is designed and configured to open a glass ampule within the container by bending the container relative to the bottom part, or by moving a carrier against a projection.

A tenth embodiment relates to a device according to any one of the preceding embodiments, wherein the base part is configured to enable a monomer liquid to be transferred from the container into the collection region by free gravity flow.

An eleventh embodiment relates to a device according to any one of the preceding embodiments, wherein the container is arranged at an angle of from 20° to 80°, preferably 21° to 55°, with respect to the bottom part.

A twelfth embodiment relates to a device according to any one of the preceding embodiments, wherein the cartridge is detachably connectable to the base part.

A thirteenth embodiment relates to a device according to any one of the preceding embodiments, wherein the base part comprises a line element, wherein the line element is configured to establish a fluid-conducting connection between the collection region and the hollow mandrel.

A fourteenth embodiment relates to a device according to any one of the preceding embodiments, wherein the base part comprises a line element, wherein the line element is configured to establish a fluid-conducting connection between the collection region and the hollow mandrel.

A fifteenth embodiment relates to a device according to any one of the preceding embodiments, wherein the collection region comprises a recess, and wherein the hollow mandrel is preferably connected to the deepest point of the recess in order to conduct fluid.

A sixteenth embodiment relates to a device according to any one of the preceding embodiments, wherein the device is designed and configured to transfer a monomer liquid from the base part into the cartridge while the base part is placed on a support such that the bottom part is oriented toward the support.

### BRIEF DESCRIPTION OF THE DRAWINGS

FIG. 1 shows the cross-section of a base part of a device according to the invention;

FIG. 2 shows the cross-section of a cartridge of a device according to the invention;

FIG. 3 shows a glass ampule being broken open in a base part;

FIG. 4 shows monomer liquid being transferred from a base part into a cartridge;

FIG. 5 shows a base part having an ampule breaker, which comprises a movable carrier and a projection;

FIG. 6 shows a base part according to FIG. 5, in which monomer liquid is transferred from broken-open ampules into the collection region;

FIG. 7 shows a base part that is connected to a cartridge;

FIG. 8 shows a cartridge during dispensing of a bone cement dough; and,

FIG. 9 shows an isometric view of a device according to the invention, wherein the base part is separated from the cartridge.

### DETAILED DESCRIPTION

With respect to the embodiments described herein, the elements of which “have,” “contain,” or “comprise” a particular feature (for example, a material), in principle, a further embodiment is always contemplated in which the relevant element consists solely of the feature, i.e., does not comprise any other constituents. The words, “comprise” or “comprising,” are used herein synonymously with the words, “contain,” “containing,” “have,” or “having.”

In one embodiment, if an element is denoted by the singular, an embodiment is also contemplated in which more than one such element is present. The use of a term for an element in the plural in principle also encompasses an embodiment in which only a single corresponding element is included.

Unless otherwise indicated or clearly excluded from the context, it is possible in principle, and is hereby clearly contemplated, that features of different embodiments may also be present in the other embodiments described herein. Likewise, all the features described herein in connection with a method are in principle also considered to be applicable to the products, devices, kits and uses described herein, and vice versa. All such considered combinations are not explicitly listed in all instances, simply in order to keep the description brief. Technical solutions known to be equivalent to the features described herein are also intended in principle to be encompassed by the scope of the invention.

A first embodiment relates to a device for producing bone cement, comprising a base part which comprises a container for receiving a monomer liquid, a bottom part with a collection region, and a hollow mandrel with a tip, and a cartridge for receiving a PMMA bone cement powder, wherein the cartridge comprises a septum which delimits the cartridge toward the outside, wherein the device is designed and configured to form a fluid-conducting connection between the base part and the cartridge by the hollow mandrel piercing the septum so as to transfer a monomer liquid from the collection region into the cartridge.

The device according to the invention can comprise two parts that can be connected to one another, specifically a base part and a cartridge. The base part comprises a container for receiving a monomer liquid. The container may comprise a carrier for receiving a glass ampule or a pouch. Such glass ampules or pouches are used as commercially available packaging for monomer liquid of a PMMA bone cement. The container can be pre-filled with monomer liquid, which is, for example, packaged in such a glass ampule or pouch in a gas-tight manner. Alternatively, the container may be empty and fillable with monomer liquid by the user, for example by inserting a glass ampule containing monomer liquid into the container. The cartridge can also be empty or pre-filled with PMMA bone cement powder. The container may comprise a transparent region. The collection region may also comprise a transparent region. This may allow a user of the device to visually verify from the outside that all of the monomer liquid has drained from the container into the collection region. Furthermore, the base part can have a fill level marking.

The base part can be designed for placement on a support. The base part has a bottom part, which may have a flat outer surface. The bottom part comprises a collection region. The collection region is preferably designed and configured to receive monomer liquid. For example, the base part can be configured to release monomer liquid from an item of packaging, such as a glass ampule or a film pouch, and to transfer it into the collection region by gravity flow. The collection region may comprise a recess, for example a concave curvature, similar to a watch glass dish. The cross-section of the recess can be U-shaped or V-shaped. The recess may be funnel-shaped.

The capacity of the collection region for receiving monomer liquid preferably exceeds the volume of the monomer liquid contained or receivable in the container. For example, the collection region may have a volume which is at least 1.1 times the volume of the monomer liquid contained or receivable in the container. For example, if the container comprises a carrier for receiving commercially available glass ampules containing 10 ml of monomer liquid, the collection region preferably will have a volume of at least 11 ml. This applies, mutatis mutandis, to embodiments which comprise a carrier for receiving glass ampules containing 5 ml, 20 ml or 30 ml of monomer liquid.

The base part also has a hollow mandrel with a tip. The hollow mandrel preferably has an interior that allows a liquid to flow through the hollow mandrel.

The interior can have a diameter of 1.0 mm to 3.0 mm. The hollow mandrel can comprise outlet openings with a diameter of less than 1.5 mm and particularly preferably less than 1.0 mm. Due to the small diameter of the hollow mandrel, a sharp jet can be formed when a monomer liquid is transferred from the collection region into the cartridge, which jet can displace smaller particles of bone cement powder, if necessary. In this way, clogging of the hollow mandrel can be avoided. The hollow mandrel can have a beveled tip, similar to a ground cannula, wherein an outlet opening of the hollow mandrel is preferably located in a bevel of the tip. The hollow mandrel can have a round tip. One or more openings can be arranged near the tip, which are connected to the interior of the hollow mandrel. Furthermore, the interior of the cavity can comprise a shoulder on which a porous pin permeable to liquids is supported. This pin can serve as a filter, as described herein below. The interior of the hollow mandrel can be tapered toward the shoulder. The shoulder may be configured to hold the pin in the hollow mandrel. The hollow mandrel can be made of a plastics material or metal, e.g. stainless steel. Examples of usable plastics include nylon 12, nylon 6, nylon 66 and polyethersulfone. The hollow mandrel, in particular the tip of the hollow mandrel, may be covered by a removable protective cap.

The cartridge comprises a septum which delimits the cartridge toward the outside. The septum is preferably arranged in a region of the cartridge which is designed and configured for connection to the base part. The septum can be pierced by the hollow mandrel of the base part. In a configuration of the device in which the septum is pierced by the hollow mandrel, a fluid-conducting connection can form between the base part and the cartridge. This makes it possible to transfer a monomer liquid from the collection region of the base part into the cartridge. The self-sealing function of the septum can prevent monomer liquid from leaking out of the device. This also applies if the hollow mandrel is pulled out of the septum again, as the puncture site can close automatically as a result of the restoring force of the septum. Furthermore, this can prevent contamination from entering the device from the outside.

In some embodiments, the septum comprises a flexible, self-sealing polymer. The polymer may comprise a polyhalogenated olefin, a silicone, rubber, butyl rubber, ethylene propylene diene rubber (EPDM), or a similar elastomer. The septum is preferably configured to seal the cartridge in a liquid-tight manner after the septum has been pierced by the hollow mandrel. The septum is preferably stretchable and exhibits rubber-elastic properties, as is known, for example, from septa in chemical bottles. This allows the septum to heal itself after being punctured, i.e., to automatically seal.

The septum preferably comprises a biocompatible material. The septum can comprise a circular shape. In one embodiment, the septum comprises a material which can be pierced with a medical injection cannula according to ISO 7864:2016 with a force of less than 100 N. The septum is preferably connected to the cartridge in a liquid-tight manner. In one embodiment, the septum is designed and configured to seal a puncture site in the septum in a liquid-tight manner after a puncturing by and subsequent removal of the hollow mandrel. This can be achieved by the restoring force of a rubber-elastic material of the septum.

The cartridge may comprise a guide element which facilitates insertion of the hollow mandrel into a desired position of the septum, e.g., an insertion bevel. The septum can be mounted in a holder. The holder can be detachably connected to the cartridge. The holder of the septum may be designed as a piston. This piston may be designed and configured to dispense bone cement dough from the cartridge. The holder for the septum may be arranged in a cartridge head. The cartridge head may be designed as a releasable cartridge closure.

The cartridge and the base part can preferably be stored separately from one another, i.e., they are designed as separate sub-elements that can be releasably connected to one another. This can prevent fatigue of the septum's restoring force during storage and facilitate automatic closure of the septum. Unwanted leakage of a monomer liquid from the cartridge after removal of the hollow mandrel from the septum can be prevented. Furthermore, separate storage has the advantage that packaging materials of small dimensions can be used.

In one embodiment, the device further comprises a piston. The piston can be arranged within the base part. The piston may have a passage for receiving the hollow mandrel. The passage can extend from an interior of the base part to the outside. In a basic configuration of the device, the piston may be configured to receive the tip of the hollow mandrel in the passage. In such a basic configuration of the device, no external force is exerted on the device. In this state, the tip of the hollow mandrel is arranged inside the piston such that a user is protected from injury resulting from contact with the tip. In one embodiment, the piston is configured to release the tip of the hollow mandrel from the passage when the cartridge is pushed onto the piston. This allows the cartridge to apply pressure to the piston in order to release the hollow mandrel and pierce the septum of the cartridge. The tip of the hollow mandrel can project from the passage. In order to receive a monomer liquid in the cartridge, the cartridge and base part can therefore be connected to one another in a fluid-tight manner with no risk of injury. The piston is preferably configured to be inserted through the cartridge into the base part when the cartridge is connected to the base part. As a result of the cartridge applying pressure to the piston, the piston can therefore be moved.

In one embodiment, the base part comprises a spring which is arranged and configured for movably mounting the piston within the base part. This allows the piston to be held in a position in which the tip of the hollow mandrel is received in the passage of the piston, as described above, and is thus held inside the piston without any risk of injury. The spring can be a spiral spring. In one embodiment, the spring surrounds the hollow mandrel in a portion of the hollow mandrel that is located outside the passage of the piston. The spring can be arranged between the bottom part and the piston.

In an embodiment in which the hollow mandrel is mounted in a cylindrical holder, as described herein, the spring can be arranged between this holder and the piston. The spring can facilitate the separation of the base part and the cartridge. Furthermore, the tip of the hollow mandrel can be automatically moved back into the cavity by the spring as soon as no more external forces are acting on the piston, e.g. when the user is no longer actively pushing the cartridge against the base part.

In one embodiment, the base part comprises a stop. The stop may be designed and configured to limit the movement of the piston and/or of the cartridge in the base part. The stop may be configured to hold the piston in the base part. In an embodiment in which the base part comprises a spring, the stop can limit the travel of the piston in the direction of the force applied by the spring. This can prevent the piston from being pushed out of the base part by the spring. In one embodiment, the stop is configured to hold the piston in a basic configuration of the device in a position in which the piston ends flush with an outer surface of the base part. In one embodiment, the stop is configured to hold the piston in a position in which the tip of the hollow mandrel is received in the passage of the piston. Furthermore, the stop can be designed and configured to limit the travel of the cartridge into the interior of the base part. This allows the cartridge to be placed in a desired position on the base part without the cartridge and base part moving with respect to one another. A detachable connecting element may be provided to hold the cartridge on the base part in a desired position. In such a desired position, the cartridge can end flush with the stop. This connecting element can prevent the spring from pushing the cartridge out of the base part. In such an embodiment, in order to hold the cartridge in position, the user thus does not have to compensate for the spring pressure with their own strength when using the device. The cartridge may comprise a projection designed and configured to engage in the stop.

In one embodiment, the device comprises a filter. The filter can be arranged in the base part. For example, the filter can be arranged between the container and the hollow mandrel. The filter is preferably designed and configured to prevent particles from the base part entering the cartridge. For example, the filter may be arranged and configured to retain glass fragments from a glass ampule broken open in the base part. The filter can be, for example, a weave made of metal wire or a mesh made of a plastics fabric. The filter can also be a disk made of metal or plastics material and having expediently dimensioned holes. Furthermore, open-pore foams, such as Porex, can be used. Preferably, the filter should be made of a material which is not attacked by the monomer liquid held in a receivable glass ampule.

In one embodiment, the cartridge has a connection for connection to a vacuum source. Such a connection can enable a monomer liquid to be transferred from the collection region into the cartridge by means of a pressure difference. For example, by means of a vacuum pump that can be connected to the cartridge, a negative pressure can be created in the cartridge in order to draw monomer liquid from the base part and into the cartridge through the fluid-conducting connection, which can be established by the hollow mandrel piercing the septum. The connection is preferably spaced apart from the septum such that no monomer liquid can enter the connection, in particular in a configuration in which the base part is placed on a support. For example, the septum and connection can be arranged at opposite ends of the cartridge.

In one embodiment, the container comprises an ampule breaker. The ampule breaker may be designed to open a glass ampule within the container. The container may also comprise another means for releasing a monomer liquid from an item of packaging, e.g., from a film pouch. This can, for example, be a mandrel that is suitable for piercing a film pouch.

In one embodiment, the ampule breaker is designed and configured to open a glass ampule inside the container by bending the container relative to the bottom part. For this purpose, the container may comprise a flexible housing part which enables a force to be exerted on a glass ampule in the container. The ampule breaker may comprise a rigid housing part or projection against which a glass ampule can be pushed to break it open.

In one embodiment, the base part is configured to enable a monomer liquid to be transferred from the container into the collection region by means of free gravity flow. “Free gravity flow” means here that a monomer liquid can pass from the container into the collection region by the action of gravity alone, without the need for, for example, a pressure difference between the container and the collection region for this purpose. Preferably, the devices described herein allow complete transfer of the monomer liquid from the container into the collection region by free gravity flow within 20 seconds. The monomer liquid can then be transferred from the collection region into the cartridge by suction, as described herein.

In one embodiment, the container is arranged at an angle to the bottom part of from 20° to 80°, preferably 21° to 55°. This angle is defined by the inside of each container and bottom part, where the container and bottom part merge into one another in the direction of the bottom part (i.e., in the direction of the “lower side” of the base part) at a vertical side wall of the bottom part. This corresponds to the point at which the monomer liquid flows from the container into the collection region when the device is used as intended. The container is preferably arranged opposite the bottom part, i.e. oriented “upward” when the device is used as intended, in order to allow the monomer liquid to flow into the collection region. For example, the container can be arranged in relation to the rest of the base part in a manner similar to a wing of a wing nut. The base part can, for example, comprise two or more containers. In one embodiment, the base part comprises two containers arranged in a Y shape. The piston can be arranged between the containers, i.e., between the two Y-shaped arms. The container may be arranged such that, in a configuration of the base part in which the base part is placed on a support with the bottom part oriented toward the support, it is located above the collection region. This allows reliable drainage of monomer liquid from the container into the collection region.

In one embodiment, a longitudinal axis of the container is arranged at an angle of from 20° to 80°, preferably 21° to 55°, to an axis that runs perpendicularly to a supporting surface of the bottom part. With an angle of 21° to 55°, the formation of a meniscus by the monomer liquid can in particular be prevented.

In one embodiment, the cartridge is detachably connectable to the base part. For this purpose, appropriate connecting elements, such as a thread, can be provided on the cartridge and the base part. The connecting elements can be arranged in such a way that they allow the hollow mandrel to be connected to the septum. Furthermore, the device can comprise a guide element which facilitates the connection of the base part to the cartridge in a desired orientation. Preferably, the base part and the cartridge can be connected to one another in such a way that the piston and the septum are flush with one another. For this purpose, for example, the base part can comprise a projecting region which is designed and configured to engage in the cartridge.

In one embodiment, the base part has a line element, wherein the line element is configured to establish a fluid-conducting connection between the collection region and the hollow mandrel. The line element enables monomer liquid to be transferred from the collection region into the hollow mandrel, preferably by means of free gravity flow. The line element can be designed, for example, as a hose, tube or passage. In an embodiment in which the hollow mandrel is mounted in a cylindrical holder, as described herein, the line element can be designed as a passage in the holder.

In one embodiment, the device is designed and configured to transfer a monomer liquid from the base part into the cartridge while the base part is placed on a support such that the bottom part is oriented toward the support. The containers and the cartridge preferably point “upward”, i.e., in the opposite direction to the support and against the direction of gravity. The monomer liquid can therefore flow from the container into the collection region by the effect of gravity.

In some embodiments, the cartridge comprises a PMMA bone cement powder. The PMMA bone cement powder is preferably arranged in an interior of the cartridge.

In some embodiments, the container comprises a monomer liquid. The monomer liquid is preferably arranged in an interior of the container. The monomer liquid may be arranged in a glass ampule or in a sealed film pouch within the container.

In some embodiments, the container further comprises a filter that is permeable to a monomer liquid. The filter is preferably arranged and configured to prevent glass splinters from the container from entering the cartridge. The device can be designed to receive a glass ampule containing monomer liquid. The device can further be designed to break open a glass ampule containing monomer liquid. The glass ampule can be received and broken open, in particular inside the container. For this purpose, the container can comprise a receptacle for a glass ampule. The container may comprise a means for breaking open a glass ampule. For example, the container may comprise a projection arranged and configured to break off the head of a glass ampule.

The filter may be designed to retain fragments of a glass ampule in the container. This can prevent glass splinters from entering the cartridge. This can prevent contamination of bone cement dough with glass splinters.

In some embodiments, the device comprises a mixing rod. The mixing rod is preferably designed for mixing monomer liquid and PMMA bone cement powder. The mixing rod may comprise blades. The blades can facilitate homogeneous mixing of monomer liquid and PMMA bone cement powder. The blades are preferably arranged at an end of the mixing rod which is arranged inside the cartridge. The mixing rod may comprise one, two or more than two blades. The mixing rod may comprise a handle at one end of the mixing rod which is preferably arranged outside the cartridge. The mixing rod is preferably movably arranged in the cartridge.

The mixing rod may comprise a discharge tube. Here, the discharge tube can be designed and configured to dispense a bone cement dough mixed in the device (i.e., a bone cement dough that has been mixed from monomer liquid and PMMA bone cement powder). Preferably, the discharge tube is closable. This can prevent bone cement dough from accidentally escaping from the cartridge during mixing. The discharge tube can be designed as a cavity within the mixing rod. In some embodiments, the discharge tube can be closed by a removable core. Accordingly, the mixing rod can have a removable, rod-shaped core, for example a metal core. The core can also improve the strength of the mixing rod.

The mixing rod may have a predetermined breaking point to allow the mixing rod to be broken off after the mixing process. The predetermined breaking point is preferably located outside the cartridge such that the mixing rod can be broken off from the outside without the cartridge being opened.

In some embodiments, the container further comprises a carrier for receiving an ampule. Such ampules can in particular be glass ampules in which monomer liquid is commercially available. The carrier is preferably arranged and configured to allow an ampule to be broken open and monomer liquid to flow out of the ampule through the outlet and the discharge tube by means of free gravity flow.

In some embodiments, the container further comprises a means for opening a film pouch or glass ampule within the device. An example of a means for opening a film pouch is a mandrel which is suitable for piercing a film pouch. Such a mandrel can be made, for example, from a rigid plastics material or from metal. An example of a means for opening a glass ampule is a projection against which the head of a glass ampule can be pushed to break it open. A means for opening a glass ampule may comprise a flexible housing part such that the glass ampule can be broken from the outside by hand by deforming the flexible housing part. A means for opening a glass ampule may also comprise a movably arranged element, such as a piston, which can push the glass ampule against a hard housing part, e.g. a projection, and thereby break it open. Means for opening a film pouch or a glass ampule are further disclosed in DE19532015A1, WO9718031A1, EP2404864B1 and WO2010012114A1.

In one embodiment, the container has a projection arranged in the container, which is designed and configured to break open a glass ampule that can be received in the container, and further has a carrier movably arranged in the container for receiving a glass ampule, wherein the carrier is designed and configured to break open a glass ampule that can be received in the holder by pushing the glass ampule against the projection. The carrier can, for example, be designed as a movable piston.

In one embodiment, the container has a flexible region and a rigid region, wherein the flexible region is configured to move a glass ampule received in the container against the rigid region in order to break open the glass ampule.

Furthermore, the base part may comprise a ventilation opening. The ventilation opening can be arranged in the container. The ventilation opening can preferably be arranged above the collection region in the base part. Preferably, the ventilation opening is arranged in the base part in such a way that contact with monomer liquid in the collection region is avoided. The ventilation opening can be designed either as a simple opening or as a valve. The opening can be covered on the inside by a gas-permeable porous material so as to prevent particles from entering the container from the outside and at the same time allowing air to be exchanged through the porous material. The valve can, for example, be designed to allow air to pass only in the direction of the interior of the container. This can improve the transfer of the monomer liquid into the cartridge, especially when the transfer is effected by a pressure difference. Furthermore, a unidirectional valve can prevent unwanted escape of monomer liquid from the device.

A further aspect relates to a method for producing a bone cement dough, the method comprising the following steps of providing a device according to any one of the embodiments described herein; optionally introducing a monomer liquid into the container; optionally introducing a PMMA bone cement powder into the cartridge; optionally releasing the monomer liquid from a packaging in the container; arranging the device so that the monomer liquid is transferred from the container into the collection region by free gravity flow; bringing the cartridge into contact with the base part; inserting the hollow mandrel through the septum so that a fluid-conducting connection is formed between the collection region and the cartridge; transferring the monomer liquid from the container into the cartridge; mixing the monomer liquid with the PMMA bone cement powder in the cartridge to obtain a bone cement dough.

A further aspect relates to a method for producing a bone cement dough, the method comprising the following steps of providing a device comprising a base part containing monomer liquid, a movable piston comprising a passage and a hollow mandrel, and a cartridge comprising a septum and a PMMA bone cement powder; pushing the cartridge against the piston so that the tip of the hollow mandrel is released from the passage of the piston; inserting the hollow mandrel through the septum so that a fluid-conducting connection is formed between the collection region and the cartridge; transferring the monomer liquid from the container into the cartridge; mixing the monomer liquid with the PMMA bone cement powder in the cartridge to obtain a bone cement dough.

A further aspect relates to the use of a device as described herein or of the method described above for producing bone cement dough, in particular PMMA bone cement dough. Here, a monomer liquid can be mixed with a PMMA bone cement powder within the device to form a bone cement dough. Such a bone cement dough can be dispensed from the device and hardened into PMMA bone cement at a target location.

### EXAMPLES

The invention is further illustrated below using examples which are, however, not to be understood as limiting. It will be apparent to a person skilled in the art that other equivalent means may be used similarly in place of the features described here.

### DETAILED DESCRIPTION OF THE INVENTION

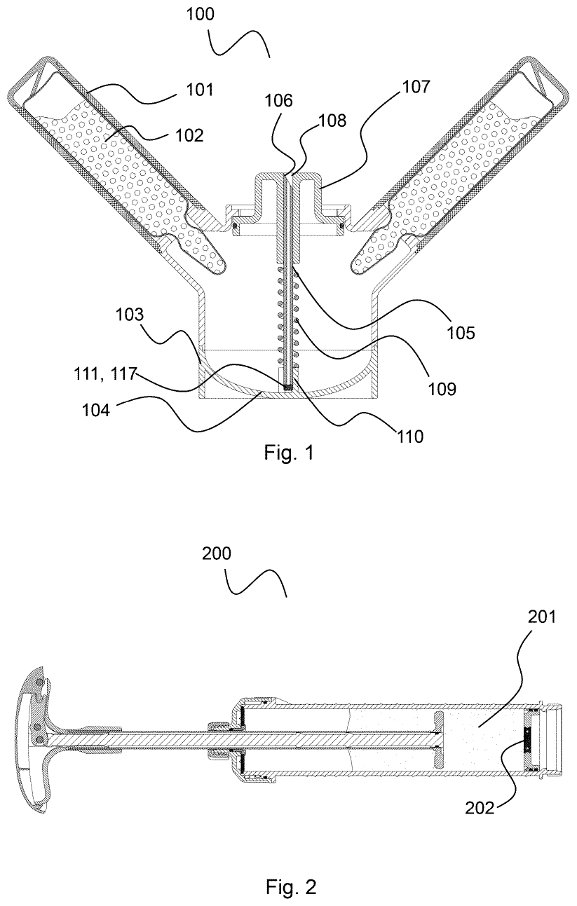

FIG. 1 shows an example of an embodiment of a base part 100 of a device according to the invention. The base part 100 comprises a container 101 which is designed for receiving a monomer liquid 102. The container 101 further comprises a bottom part 103 which is designed and configured to place the base part 100 on a support. For this purpose, the bottom part can have a flat outer surface and/or one or more foot elements. Inside the bottom part 103 a collection region 104 is arranged. The collection region 104 is designed to receive a monomer liquid which can be dispensed from the container 101. Within the base part 100 a hollow mandrel 105 is arranged which extends from the collection region 104 to an end opposite the collection region. A piston 107 is also arranged in the base part 100, which piston is movably mounted on a spring 109. The piston has a passage 108 which is designed as a cylindrical cavity. The passage is designed and configured to receive the hollow mandrel 105. The hollow mandrel has a tip 106 which is received in the cavity in a basic state of the base part. The hollow mandrel 105 is mounted in a holder 110 at an end of the hollow mandrel that is opposite the tip 106, which holder is arranged in the bottom part 103. The holder 110 has a line element 111 which here is designed as an aperture through the holder 110. The line element 111 establishes a fluid-conducting connection between the collection region 104 and the hollow mandrel 105. A filter 117 which can prevent glass fragments from entering the hollow mandrel 105 from the collection region 104 is arranged on the line element 111.

FIG. 2 shows a cartridge 200 of a device according to the invention. The cartridge 200 is designed and configured to receive a PMMA bone cement powder 201. The cartridge 200 has a septum 202 which delimits the cartridge toward the outside. The septum 202 is connected to the cartridge housing in a liquid-tight manner. The cartridge is designed and configured to be brought into contact with the base part 100 in order to receive in the cartridge a monomer liquid from the base part 100.

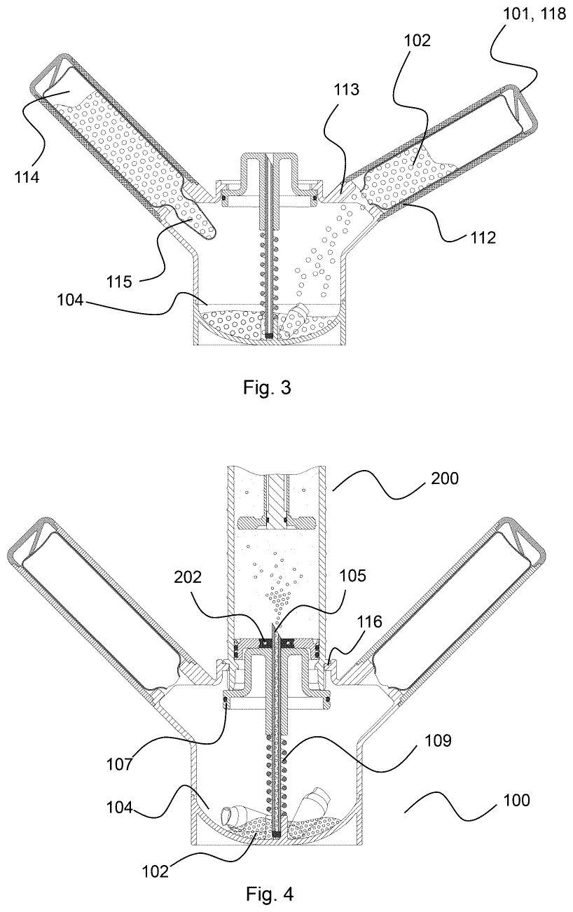

FIG. 3 shows a base part 100 having a container 101, which comprises a carrier 118 for receiving a glass ampule 114. The container 101 has a flexible region 112 and a rigid region 113. A user can bend the flexible region 112 to push the ampule head 115 of the glass ampule 114 received in the container 101 against the rigid region 113 in order to break the ampule head 115 off and thereby release the monomer liquid 102 from the glass ampule 114. The monomer liquid 102 can flow into the collection region 104 by free gravity flow.

FIG. 4 shows a base part 100, which is brought into contact with a cartridge 200. The hollow mandrel 105 is guided through the septum 202 to form a fluid-conducting connection between the collection region 104 and the interior of the cartridge 200. Monomer liquid 102 can thus be transferred from the collection region 104 into the cartridge 200, for example by connecting a vacuum source to the cartridge 200. The base part 100 comprises a stop 116 which is designed to engage in a projection on the cartridge 200. By pushing the cartridge 200 against the piston 107, the piston is moved into the interior of the base part 100, this movement being limited by the stop 116. The stop 116 can further limit the movement of the piston 107 to the outside in order to prevent the spring 109 from moving the piston 107 fully out of the base part 100.

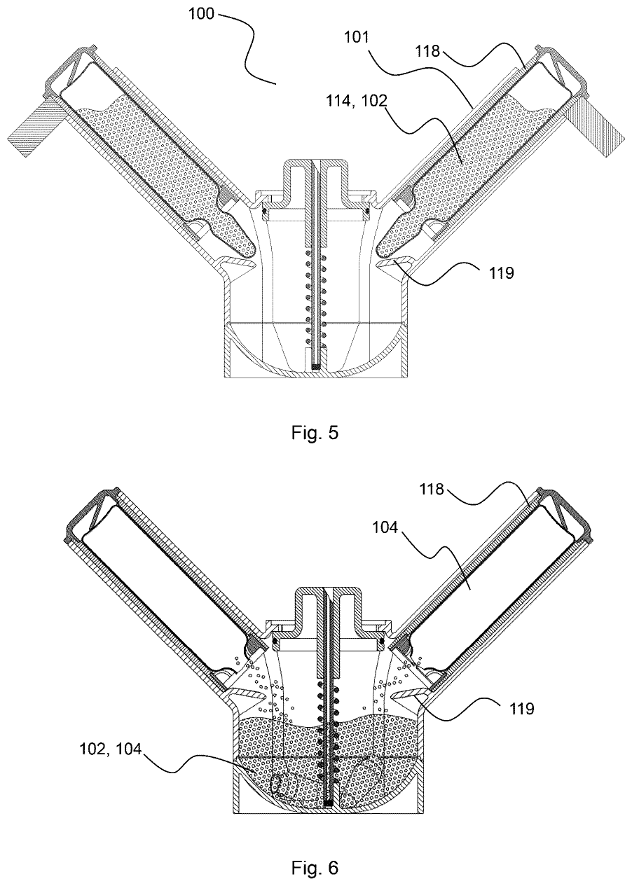

FIG. 5 shows a base part 100 comprising a container 101, which comprises a carrier 118 for receiving a glass ampule 114. The carrier 118 is movably arranged in the container 101. The container 101 also comprises a projection 119. By means of the movable carrier 118, a glass ampule 114 can be pushed against the projection 119 to break open the glass ampule and release a monomer liquid 102 from the glass ampule.

FIG. 6 shows a base part 100 according to FIG. 5, wherein a glass ampule 114 is broken open by pushing the movable carrier 118 against the projection 119 in order to dispense a monomer liquid 102 into the collection region 104.

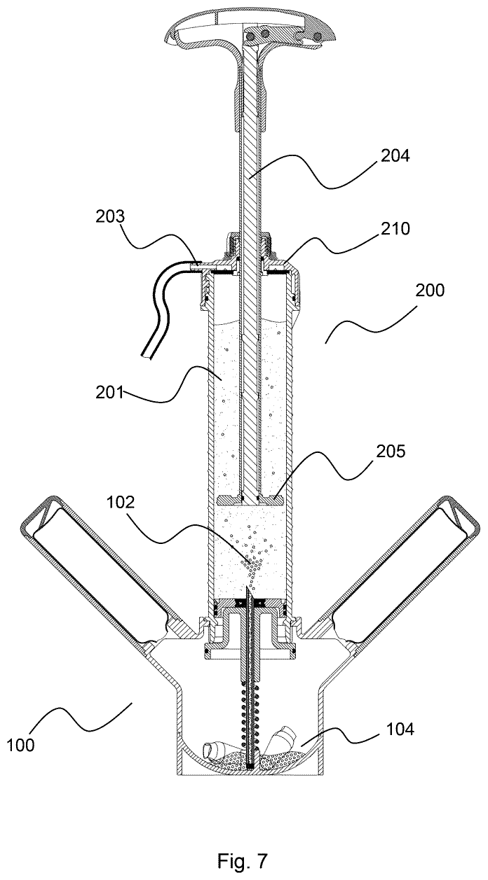

FIG. 7 shows a device according to the invention, wherein the cartridge 200 comprises a connection 203 for a vacuum source. This can create a negative pressure in the cartridge 200 in order to transfer a monomer liquid 102 from the collection region 104 of the base part 100 into the cartridge 200. The cartridge contains a PMMA bone cement powder 201, which can be mixed with the monomer liquid 102 to form a bone cement dough. For this purpose, the cartridge 200 further comprises a mixing rod 204 with a mixing disk 205. The mixing rod 204 is guided through a cartridge head 210 screwed onto the cartridge 200. The connection 203 is arranged on the cartridge head 210. The mixing disk 205 is arranged at an end of the mixing rod 204 which is arranged inside the cartridge 200.

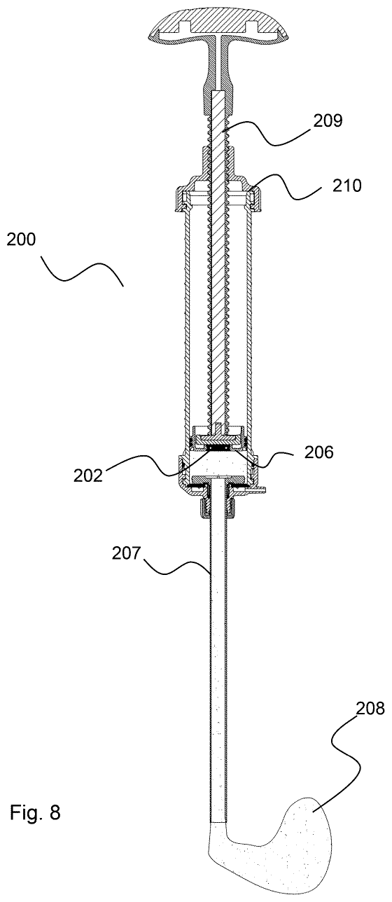

FIG. 8 shows a cartridge 200 comprising a threaded rod 209. The threaded rod 209 engages in a cartridge head 210 in order to be able to screw the threaded rod 209 into the cartridge. This allows a holder 206 for the septum 202 to be moved inside the cartridge 200 in the manner of a piston in order to dispense bone cement dough 208 mixed in the cartridge from the cartridge 200 through a discharge tube 207.

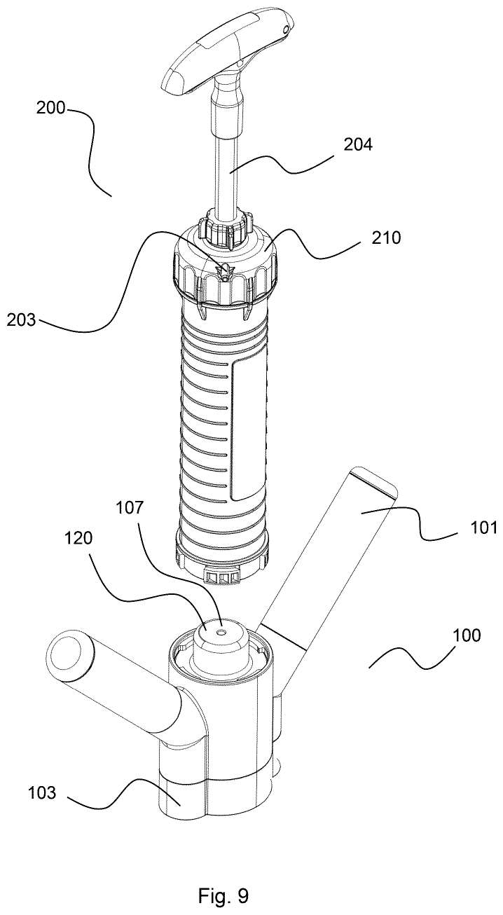

FIG. 9 shows an embodiment of a device according to the invention, which comprises a base part 100 and a cartridge 200 that can be connected thereto. The base part 100 has a screw thread for connecting to the cartridge 200. The base part 100 also comprises an outwardly projecting contact region 120 which can be received in the cartridge 200. A piston 107 and a hollow mandrel 105 are arranged in this contact region 120. The base part 100 also has a bottom part 103 which can be placed on a support during use of the device. The base part 100 further comprises a container 101 for receiving glass ampules containing monomer liquid. The container 101 has a closable opening for receiving a glass ampule. The cartridge 200 comprises a cartridge head 210 having a vacuum connection 203. The cartridge 200 also comprises a mixing rod 204 which is guided through the cartridge head 210 and into the interior of the cartridge 200.

### LIST OF REFERENCE NUMERALS

100 base part
101 container
102 monomer liquid
103 bottom part
104 collection region
105 hollow mandrel
106 tip
107 piston
108 passage
109 spring
110 holder for hollow mandrel
111 line element
112 flexible region of the container
113 rigid region of the container
114 glass ampule
115 ampule head
116 stop
117 filter
118 carrier
119 projection
120 contact region
200 cartridge
201 PMMA bone cement powder
202 septum
203 connection for vacuum source
204 mixing rod
205 mixing disk
206 septum holder
207 discharge tube
208 bone cement dough
209 threaded rod
210 cartridge head

1. A device for producing bone cement, comprising:
a base part (100) which comprises a container (101) for receiving a monomer liquid (102), a bottom part (103) having a collection region (104), and a hollow mandrel (105) having a tip (106), and a cartridge (200) for receiving a PMMA bone cement powder (201), wherein the cartridge comprises a septum (202) which delimits the cartridge toward the outside, wherein the device is designed and configured to form a fluid-conducting connection between the base part and the cartridge by the hollow mandrel piercing the septum so as to transfer a monomer liquid from the collection region into the cartridge.

2. The device according to claim 1, wherein the base part further comprises a piston (107), wherein the piston comprises a passage (108) for receiving the hollow mandrel, wherein, in a basic configuration of the device, the piston is configured to receive the tip of the hollow mandrel in the passage, and to release the tip of the hollow mandrel from the passage when the cartridge is pushed onto the piston.

3. The device according to claim 2, wherein the base part comprises a spring (109) which is arranged and configured for movably mounting the piston within the base part.

4. The device according to claim 2, wherein the base part comprises a stop (116) to limit the movement of the piston and/or the cartridge in the base part.

5. The device according to claim 1, wherein the device comprises a filter, wherein the filter (117) is arranged and designed to prevent particles from entering the cartridge from the base part.

6. The device according to claim 1, wherein the cartridge comprises a connection (203) for connection to a vacuum source to enable a monomer liquid to be transferred from the collection region into the cartridge by means of a pressure difference.

7. The device according to claim 1, wherein the container comprises a carrier (118) for receiving a glass ampule or a film pouch containing monomer liquid.

8. The device according to claim 1, wherein the container comprises an ampule breaker (113), wherein the ampule breaker is configured to open a glass ampule within the container.

9. The device according to claim 8, wherein the ampule breaker is designed and configured to open a glass ampule within the container by bending the container relative to the bottom part, or by moving a carrier (118) against a projection (119).

10. The device according to claim 1, wherein the base part is configured to enable a monomer liquid to be transferred from the container into the collection region by free gravity flow.

11. The device according to claim 1, wherein the container is arranged at an angle of from 20° to 80° with respect to the bottom part.

12. The device according to claim 1, wherein the cartridge is detachably connectable to the base part.

13. The device according to claim 1, wherein the base part comprises a line element (111), wherein the line element is configured to establish a fluid-conducting connection between the collection region and the hollow mandrel.

14. The device according to claim 13, wherein the hollow mandrel is mounted in a cylindrical holder (110), wherein the holder is arranged in the collection region.

15. The device according to claim 1, wherein the collection region comprises a recess, and wherein the hollow mandrel is preferably connected to the deepest point of the recess in order to conduct fluid.

16. The device according to claim 1, wherein the device is designed and configured to transfer a monomer liquid from the base part into the cartridge while the base part is placed on a support such that the bottom part is oriented toward the support.
